(19) United States
(12) Patent Application Publication
(10) Pub. No.: US 2023/0186658 A1
(43) Pub. Date: Jun. 15, 2023

(54) GENERATION DEVICE, GENERATION METHOD, GENERATION PROGRAM, AND DIAGNOSIS SUPPORT SYSTEM

(71) Applicant: SONY GROUP CORPORATION, TOKYO (JP)

(72) Inventors: JUNICHIRO ENOKI, TOKYO (JP); KENJI YAMANE, TOKYO (JP)

(21) Appl. No.: 17/996,799

(22) PCT Filed: Apr. 20, 2021
(86) PCT No.: PCT/JP2021/015946

§ 371 Date: Oct. 21, 2022

(30) Foreign Application Priority Data
Apr. 30, 2020 (JP) 2020-080843

(51) Int. Cl. G06V 20/69; G06V 10/22; G16H 30/40

(52) U.S. Cl. CPC G06V 20/695; G06V 10/22; G16H 30/40; G06V 2201/03

(57) ABSTRACT

A generation device includes: a generation section that, on the basis of feature values of partial regions set in an image regarding pathology, generates distribution information in which distributions of the partial regions on a feature space specified by feature values included in the partial regions are visibly arranged in the feature space while being associated with the partial regions.

### FIELD

The present invention relates to a generation device, a generation method, a generation program, and a diagnosis support system.

### BACKGROUND

There is a system that photographs an observation target placed on a glass slide with a microscope, generates a digitized pathological image, and performs various image analyses on the pathological image. For example, the observation target is a tissue or a cell taken from a patient, and corresponds to a piece of an organ, saliva, blood, or the like.

In a conventional system, if a user specifies an analysis target region on a pathological image, analysis is performed on the specified region by a predetermined analysis procedure, and a final analysis result is outputted. The user adjusts parameters used for analysis processing while referring to the analysis result.

### CITATION LIST

### Patent Literature

Patent Literature 1: US 2007/030529 A
Patent Literature 2: US 2009/208134 A

### SUMMARY

### Technical Problem

In the case where a user selects a plurality of analysis target regions, it is preferable to select a region having variations in characteristics, because an analysis result of a region having various characteristics can be obtained. However, a conventional system remains unable to support the selection of a region having variations in characteristics.

Thus, the present disclosure proposes a generation device, a generation method, a generation program, and a diagnosis support system that can support the selection of a region having variations in characteristics.

### Solution to Problem

To solve the problems described above, a generation device according to an embodiment of the present disclosure includes: a generation section that, on the basis of feature values of partial regions set in an image regarding pathology, generates distribution information in which distributions of the partial regions on a feature space specified by feature values included in the partial regions are visibly arranged in the feature space while being associated with the partial regions.

### BRIEF DESCRIPTION OF DRAWINGS

FIG. 1 is a diagram illustrating a diagnosis support system according to the present embodiment.

FIG. 2 is a diagram for describing imaging processing according to the present embodiment.

FIG. 3 is a diagram for describing imaging processing according to the present embodiment.

FIG. 4 is a diagram for describing the processing of generating partial images (tile images).

FIG. 5 is a diagram for describing a pathological image according to the present embodiment.

FIG. 6 is a diagram for describing a pathological image according to the present embodiment.

FIG. 7 is a diagram illustrating an example of a mode of viewing by a viewer of pathological images.

FIG. 8 is a diagram illustrating an example of a viewing history storage section included in a server.

FIG. 9A is a diagram illustrating a diagnostic information storage section included in a medical information system.

FIG. 9B is a diagram illustrating a diagnostic information storage section included in a medical information system.

FIG. 9C is a diagram illustrating a diagnostic information storage section included in a medical information system.

FIG. 10 is a diagram for describing processing of a generation device according to the present embodiment.

FIG. 11 is a functional block diagram illustrating a configuration of a generation device according to the present embodiment.

FIG. 12 is a diagram illustrating an example of a data structure of a pathological image DB.

FIG. 13 is a diagram illustrating an example of a data structure of a partial region table 142.

FIG. 14 is a diagram illustrating an example of a data structure of a characteristic distribution table.

FIG. 15 is a diagram for describing processing (2) executed by a display control section.

FIG. 16 is a diagram for describing processing (3) executed by the display control section.

FIG. 17 is a diagram for describing processing (4) executed by the display control section.

FIG. 18 is a diagram for describing processing (5) executed by the display control section.

FIG. 19 is a diagram for describing processing (6) executed by the display control section.

FIG. 20 is a diagram for describing processing (7) executed by the display control section.

FIG. 21 is a diagram for describing processing (8) executed by the display control section.

FIG. 22 is a diagram for describing processing (9) executed by the display control section.

FIG. 23 is a flowchart illustrating a processing procedure of a generation processing device according to the present embodiment.

FIG. 24 is a hardware configuration diagram illustrating an example of a computer that implements functions of a generation device.

### DESCRIPTION OF EMBODIMENTS

Hereinbelow, embodiments of the present disclosure are described in detail based on the drawings. In the following embodiments, the same parts are denoted by the same reference numerals, and a repeated description is omitted.

The present disclosure is described according to the following item order.

### Embodiments

1. Configuration of system according to present embodiment

2. With regard to various pieces of information

2-1. Pathological image

2-2. Viewing history information

2-3. Diagnostic information

3. Generation device according to present embodiment

3-1. Processing of generation device

3-2. Functional configuration of generation device

4. Processing procedure

5. Effects of generation device according to present embodiment

6. Hardware configuration

7. Conclusions

### 1. CONFIGURATION OF SYSTEM ACCORDING TO EMBODIMENT

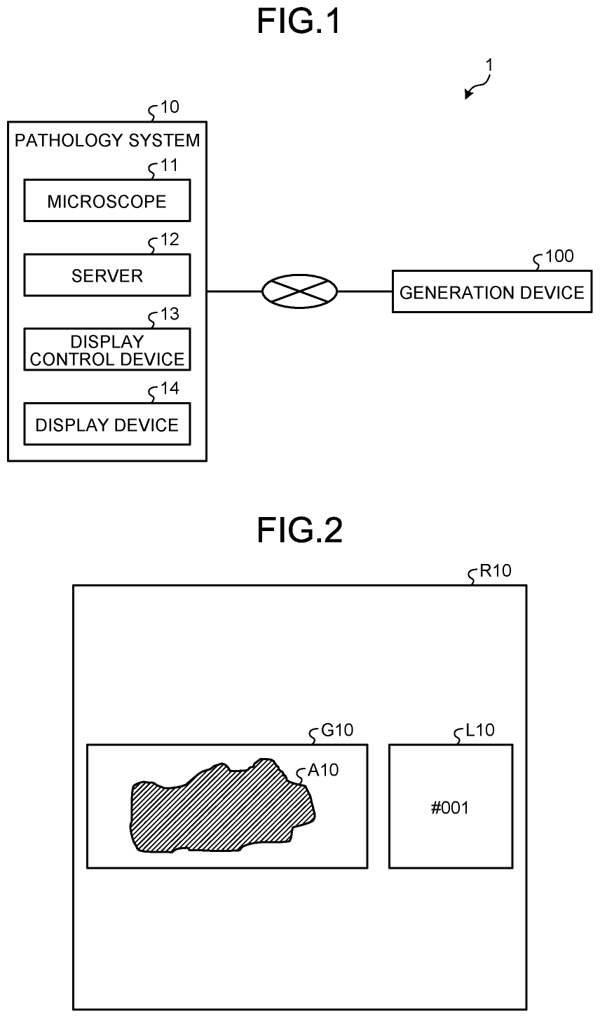

First, a diagnosis support system 1 according to a first embodiment is described using FIG. 1. FIG. 1 is a diagram illustrating a diagnosis support system 1 according to a first embodiment. As illustrated in FIG. 1, the diagnosis support system 1 includes a pathology system 10 and a generation device 100.

The pathology system 10 is a system mainly used by a pathologist, and is used for, for example, a laboratory or a hospital. As illustrated in FIG. 1, the pathology system 10 includes a microscope 11, a server 12, a display control device 13, and a display device 14.

The microscope 11 is an imaging device that has a function of an optical microscope, and images an observation target placed on a glass slide and acquires a pathological image that is a digital image (an example of a medical image). The observation target is, for example, a tissue or a cell taken from a patient, such as a piece of an organ, saliva, or blood.

The server 12 is a device that stores and saves a pathological image captured by the microscope 11 in a not-illustrated storage section. Upon accepting a viewing request from the display control device 13, the server 12 searches for a pathological image from the not-illustrated storage section, and sends the found pathological image to the display control device 13. Further, upon accepting a request to acquire a pathological image from the generation device 100, the server 12 searches the storage section for the pathological image, and sends the found pathological image to the generation device 100.

The display control device 13 sends, to the server 12, a request to view a pathological image accepted from the user. Then, the display control device 13 controls the display device 14 to display the pathological image accepted from the server 12.

The display device 14 has a screen using, for example, liquid crystals, EL (electro-luminescence), a CRT (cathode ray tube), or the like. The display device 14 may be compatible with 4K or 8K, and may be formed of a plurality of display devices. The display device 14 displays a pathological image that the display device 14 is controlled to display by the display control device 13. Although details are described later, the server 12 stores viewing history information regarding regions of a pathological image observed by a pathologist via the display device 14.

The generation device 100 sends, to the server 12, a request to acquire a pathological image, and accepts the pathological image from the server 12. Information of feature values of the pathological image is attached to the pathological image accepted from the server 12. The generation device 100 generates and displays information in which a pathological image is divided in a plurality of partial regions and distribution information in which distributions of the partial regions on a feature space specified by feature values included in the partial regions are visibly arranged in the feature space while being associated with the partial regions. A detailed description of the generation device 100 will be given in “3. Generation device according to present embodiment”.

### 2. WITH REGARD TO VARIOUS PIECES OF INFORMATION

[2-1. Pathological Image]

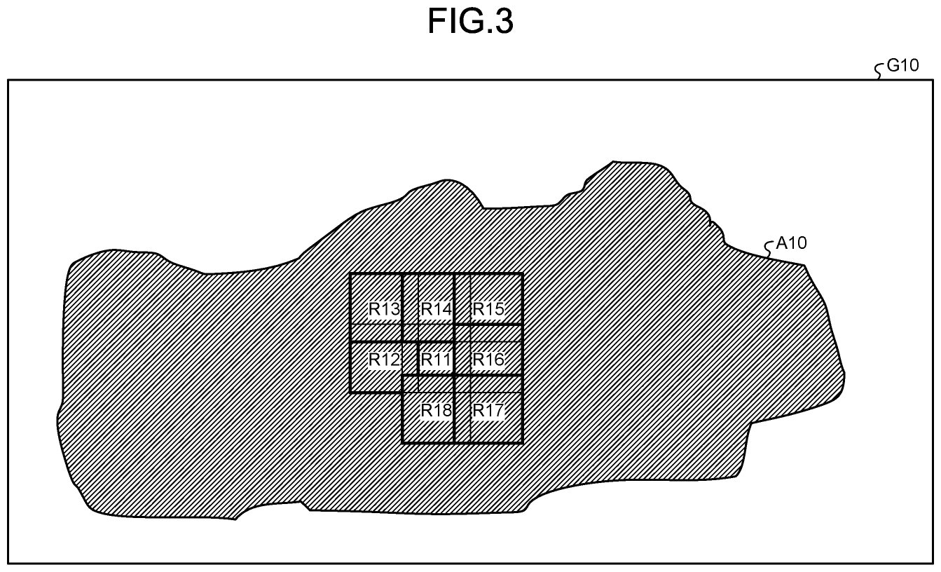

As described above, a pathological image is generated by an observation target being imaged by the microscope 11. First, imaging processing by the microscope 11 is described using FIG. 2 and FIG. 3. FIG. 2 and FIG. 3 are diagrams for describing imaging processing according to the first embodiment. The microscope 11 described below includes a low-resolution imaging section for imaging at low resolution and a high-resolution imaging section for imaging at high resolution.

In FIG. 2, a glass slide G10 containing an observation target A10 is included in an imaging region R10 that is a region that can be photographed by the microscope 11. The glass slide G10 is placed on a not-illustrated stage, for example. The microscope 11 images the imaging region R10 with the low-resolution imaging section to generate an overall image that is a pathological image in which the observation target A10 is entirely imaged. In label information L10 illustrated in FIG. 2, identification information (for example, a character string or a QR code (registered trademark)) for identifying the observation target A10 is written. The patient corresponding to the overall image can be identified by associating the identification information written in the label information L10 and the patient in advance. In the example of FIG. 2, “#001” is written as the identification information. In the label information L10, for example, a brief description of the observation target A10 may be written.

Subsequently, after the overall image is generated, the microscope 11 specifies a region including the observation target A10 from the overall image, and uses the high-resolution imaging section to sequentially image divided regions that are obtained by dividing the region including the observation target A10 in a predetermined size. For example, as illustrated in FIG. 3, the microscope 11 first images region R11, and generates high-resolution image I11 that is an image showing a partial region of the observation target A10. Subsequently, the microscope 11 moves the stage to image region R12 with the high-resolution imaging section, and generates high-resolution image 112 corresponding to region R12. Similarly, the microscope 11 generates high-resolution images 113, 114, etc. corresponding to regions R13, R14, etc. Although FIG. 3 illustrates only up to region R18, the microscope 11 sequentially moves the stage to image all the divided regions corresponding to the observation target A10 with the high-resolution imaging section, and generates high-resolution images corresponding to the divided regions.

Meanwhile, when moving the stage, the glass slide G10 may move on the stage. If the glass slide G10 moves, an un-photographed region of the observation target A10 may occur. Even if the glass slide G10 moves, the microscope 11 can prevent the occurrence of an un-photographed region by, as illustrated in FIG. 3, performing imaging with the high-resolution imaging section such that adjacent divided regions partially overlap.

The low-resolution imaging section and the high-resolution imaging section described above may be different optical systems, or may be the same optical system. In the case of the same optical system, the microscope 11 changes the resolution in accordance with the imaging target. Further, although the foregoing shows an example in which the imaging region is changed by moving the stage, the imaging region may be changed by the microscope 11 moving the optical system (the high-resolution imaging section, etc.). Further, FIG. 3 illustrates an example in which the microscope 11 performs imaging from a central portion of the observation target A10. However, the microscope 11 may image the observation target A10 in an order different from the imaging order illustrated in FIG. 3. For example, the microscope 11 may perform imaging from an outer peripheral portion of the observation target A10. Further, the foregoing shows an example in which only a region including the observation target A10 is imaged by the high-resolution imaging section. However, since there is a case where a region including the observation target A10 cannot be accurately detected, the microscope 11 may divide the entire imaging region R10 or the entire region of the glass slide G10 illustrated in FIG. 2 and image them with the high-resolution imaging section.

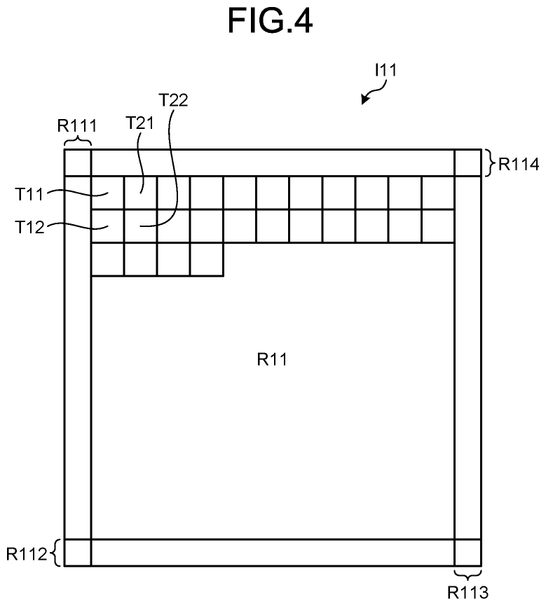

Subsequently, each high-resolution image generated by the microscope 11 is divided into a predetermined size. Thereby, partial images (hereinafter, written to as tile images) are generated from the high-resolution image. This point will now be described using FIG. 4. FIG. 4 is a diagram for describing the processing of generating partial images (tile images). FIG. 4 illustrates high-resolution image I11 corresponding to region R11 illustrated in FIG. 3. The following description is given on the assumption that partial images are generated from a high-resolution image by the server 12. However, the partial images may be generated by a device other than the server 12 (for example, an information processing device mounted inside the microscope 11, or the like).

In the example illustrated in FIG. 4, the server 12 divides one high-resolution image I11 to generate 100 tile images T11, T12, etc. For example, when the resolution of high-resolution image I11 is 2560×2560 [pixels], the server 12 generates 100 tile images T11, T12, etc. with a resolution of 256×256 [pixels] from high-resolution image I11. Similarly, the server 12 divides other high-resolution images into a similar size to generate tile images.

In the example of FIG. 4, regions R111, R112, R113, and R114 are regions overlapping with adjacent other high-resolution images (not illustrated in FIG. 4). The server 12 performs stitching processing on high-resolution images adjacent to each other by performing positioning of overlapping regions by a technique such as template matching. In this case, the server 12 may generate tile images by dividing the high-resolution image after stitching processing. Alternatively, the server 12 may generate tile images of the regions other than region R111, R112, R113, or R114 before stitching processing, and generate tile images of regions R111, R112, R113, and R114 after stitching processing.

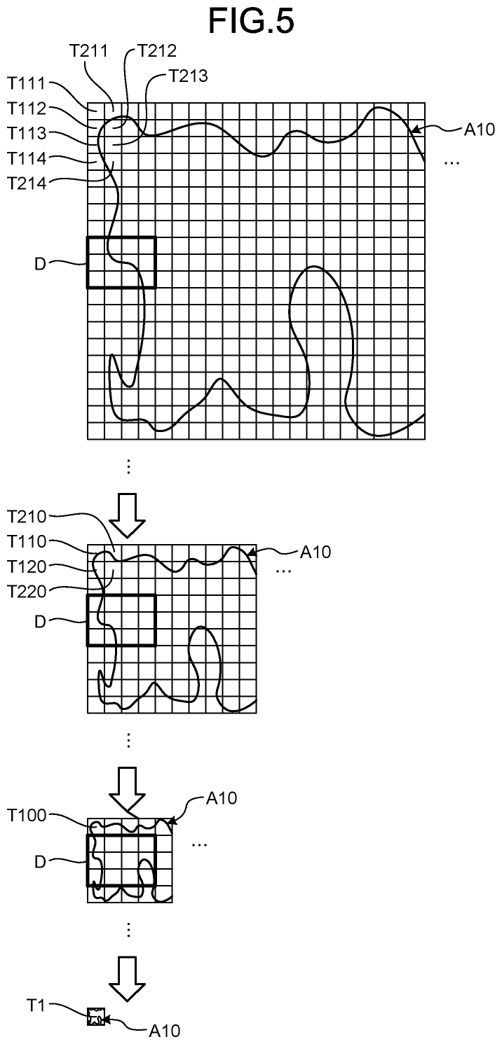

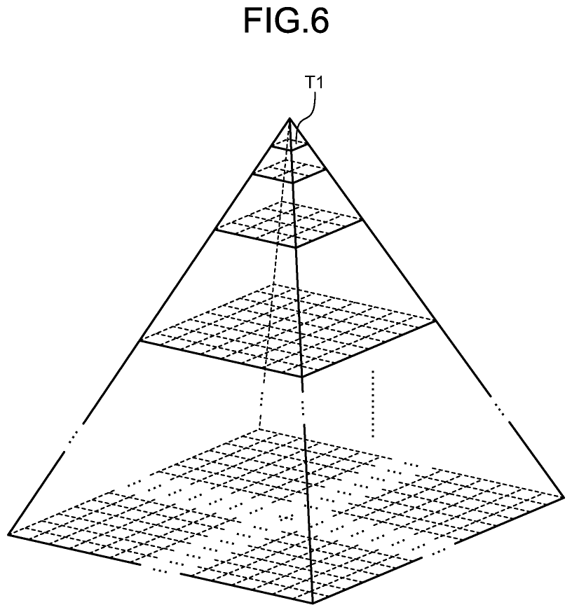

In this way, the server 12 generates a tile image that is the minimum unit of the captured image of the observation target A10. Then, the server 12 sequentially synthesizes tile images of the minimum unit, and thus generates tile images of different classes. Specifically, the server 12 synthesizes a predetermined number of adjacent tile images to generate one tile image. This point will now be described using FIG. 5 and FIG. 6. FIG. 5 and FIG. 6 are diagrams for describing a pathological image according to the present embodiment.

The upper part of FIG. 5 illustrates a tile image group of the minimum unit generated from each high-resolution image by the server 12. In the example of the upper part of FIG. 5, the server 12 synthesizes four tile images T111, T112, T211, and T212 adjacent to each other among the tile images, and thus generates one tile image T110. For example, when the resolution of each of tile images T111, T112, T211, and T212 is 256×256, the server 12 generates a tile image T110 with a resolution of 256×256. Similarly, the server 12 synthesizes four tile images T113, T114, T213, and T214 adjacent to each other, and thus generates a tile image T120. In this way, the server 12 generates tile images in each of which a predetermined number of tile images of the minimum unit are synthesized.

Further, the server 12 generates tile images in each of which, among the tile images in each of which tile images of the minimum unit are synthesized, tile images adjacent to each other are further synthesized. In the example of FIG. 5, the server 12 synthesizes four tile images T110, T120, T210, and T220 adjacent to each other, and thus generates one tile image T100. For example, when the resolution of each of tile images T110, T120, T210, and T220 is 256×256, the server 12 generates a tile image T100 with a resolution of 256×256. For example, from an image with a resolution of 512×512 in which four tile images adjacent to each other are synthesized, the server 12 generates a tile image with a resolution of 256×256 by performing processing such as four-pixel averaging, weighting filtering (processing of reflecting nearer pixels more strongly than farther pixels), or ½ thinning-out processing.

By repeating such synthesis processing, the server 12 finally generates one tile image having a resolution similar to the resolution of a tile image of the minimum unit. For example, when the resolution of a tile image of the minimum unit is 256×256 as in the above example, the server 12 finally generates one tile image T1 with a resolution of 256×256 by repeating the synthesis processing described above.

FIG. 6 schematically illustrates the tile images illustrated in FIG. 5. In the example illustrated in FIG. 6, the tile image group of the lowest layer is tile images of the minimum unit generated by the server 12. Further, the tile image group of the second lowest class is tile images after the tile image group of the lowest layer is synthesized. The tile image T1 of the highest layer indicates one tile image finally generated. In this way, the server 12 generates, as a pathological image, a tile image group having a hierarchy like the pyramid structure illustrated in FIG. 6.

Region D illustrated in FIG. 5 indicates an example of a region displayed on a display screen of the display device 14 or the like. For example, it is assumed that the resolution that the display device can perform displaying with is three vertical tile images and four horizontal tile images. In this case, like region D illustrated in FIG. 5, the degree of detail of the observation target A10 displayed on the display device varies with the class that the tile image to be displayed belongs to. For example, when the tile image of the lowest layer is used, a small region of the observation target A10 is displayed in detail. When the tile image of a higher layer is used, a larger region of the observation target A10 is displayed more roughly.

The server 12 stores tile images of various classes like those illustrated in FIG. 6 in the not-illustrated storage section. For example, the server 12 stores each tile image together with tile identification information (an example of partial image information) that can uniquely identify the tile image. In this case, upon accepting a request to acquire a tile image including tile identification information from another device (for example, the display control device 13), the server 12 transmits the tile image corresponding to the tile identification information to the other device. Further, for example, the server 12 may store each tile image together with class identification information that identifies each class and tile identification information that can make unique identification in the same class. In this case, upon accepting a request to acquire a tile image including class identification information and tile identification information from another device, the server 12 transmits, to the other device, the tile image corresponding to the tile identification information among the tile images belonging to the class corresponding to the class identification information.

The server 12 may store tile images of various classes like those illustrated in FIG. 6 in a storage device other than the server 12. For example, the server 12 may store tile images of various classes in a cloud server or the like. Further, the processing of generating tile images illustrated in FIG. 5 and FIG. 6 may be executed by a cloud server or the like.

The server 12 may not store the tile images of all the classes. For example, the server 12 may store only the tile image of the lowest layer, may store only the tile image of the lowest layer and the tile image of the highest layer, or may store only the tile image of a predetermined class (for example, odd-position classes, even-position classes, or the like). At this time, when a tile image of a class not stored is requested from another device, the server 12 generates the tile image requested from the other device by dynamically synthesizing stored tile images. Thus, the server 12 can prevent tightness of storage capacity by thinning out the tile images to be saved.

Although the above example does not mention imaging conditions, the server 12 may store tile images of various classes like those illustrated in FIG. 6 for each imaging condition. Examples of the imaging conditions include a focal distance to a subject (the observation target A10 or the like). For example, the microscope 11 may perform imaging while changing the focal distance to the same subject. In this case, the server 12 may store tile images of various classes like those illustrated in FIG. 6 for each focal distance. The reason for changing the focal distance is that, since there may be a translucent observation target A10, there is a focal distance suitable to image the surface of the observation target A10 or a focal distance suitable to image the inside of the observation target A10. In other words, by changing the focal distance, the microscope 11 can generate a pathological image in which the surface of the observation target A10 is imaged or a pathological image in which the inside of the observation target A10 is imaged.

Other examples of the imaging conditions include a staining condition for the observation target A10. Specifically, in a pathological diagnosis, a specific portion (for example, a cell nucleus or the like) of the observation target A10 may be stained with a luminescent substance. The luminescent substance is, for example, a substance that emits light when irradiated with light of a specific wavelength. There is a case where the same observation target A10 is stained with different luminescent substances. In this case, the server 12 may store tile images of various classes like those illustrated in FIG. 6 for each applied luminescent substance.

The number and resolution of tile images described above are merely examples, and may be changed depending on the system, as appropriate. For example, the number of tile images synthesized by the server 12 is not limited to four. For example, the server 12 may repeat the processing of synthesizing 3×3=9 tile images. Further, although the above example shows an example in which the resolution of the tile image is 256×256, the resolution of the tile image may be a resolution other than 256×256.

The display control device 13 uses a software application employing a system compatible with a tile image group of the hierarchical structure described above and extracts a desired tile image from a tile image group of a hierarchical structure in accordance with an input operation by the user via the display control device 13, and outputs the extracted tile image to the display device 14. Specifically, the display device 14 displays an image of an arbitrary part selected by the user among the images of an arbitrary resolution selected by the user. By such processing, the user can obtain a feeling of observing the observation target while changing the observation magnification. That is, the display control device 13 functions as a virtual microscope. The virtual observation magnification herein corresponds to the resolution in practice.

[2-2. Viewing History Information]

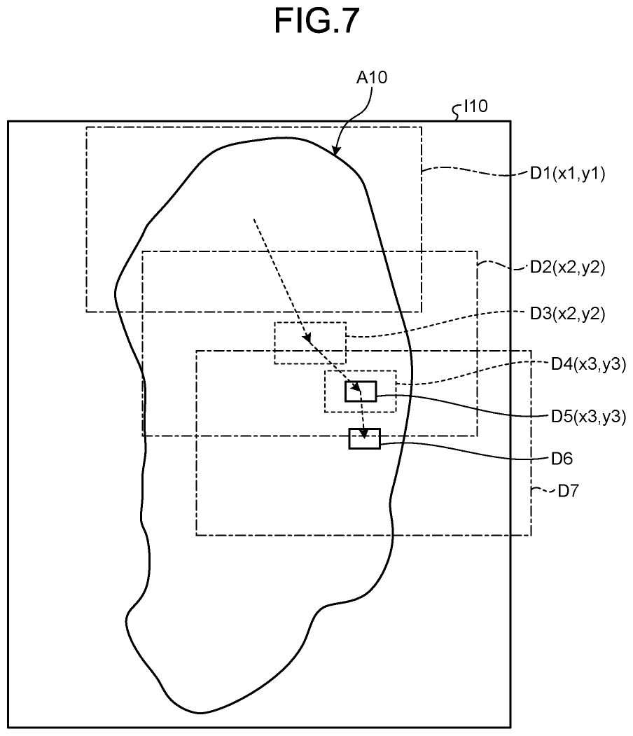

Next, information of history of viewing pathological images saved in the server 12 is described using FIG. 7. FIG. 7 is a diagram illustrating an example of a mode of viewing by a viewer of pathological images. In the example illustrated in FIG. 7, it is assumed that a viewer such as a pathologist has viewed regions D1, D2, D3, . . . , and D7 of pathological image I10 in this order. In this case, the display control device 13 first acquires a pathological image corresponding to region D1 from the server 12 in accordance with a viewing operation by the viewer. In response to a request from the display control device 13, the server 12 acquires, from the storage section, one or more tile images that form a pathological image corresponding to region D1, and transmits the acquired one or more tile images to the display control device 13. Then, the display control device 13 displays, on the display device 14, a pathological image formed from the one or more tile images acquired from the server 12. For example, when there are a plurality of tile images, the display control device 13 displays the plurality of tile images side by side. Similarly, each time the viewer performs the operation of changing the display region, the display control device 13 acquires a pathological image corresponding to the region to be displayed (regions D2, D3, . . . , D7, etc.) from the server 12, and displays the acquired pathological image on the display device 14.

In the example of FIG. 7, the viewer first views a relatively large region D1, but finds that there is no region to be carefully observed in region D1, and thus moves the viewing region to region D2. Then, the viewer finds that there is a region desired to be carefully observed in region D2, and thus enlarges a partial region of region D2 and views region D3. Then, the viewer further moves the viewing region to region D4, which is a partial region of region D2. Then, the viewer finds that there is a region desired to be observed more carefully in region D4, and thus enlarges a partial region of region D4 and views region D5. In this way, the viewer views also regions D6 and D7. For example, the pathological images corresponding to regions D1, D2, and D7 are display images magnified 1.25 times, the pathological images corresponding to regions D3 and D4 are display images magnified 20 times, and the pathological images corresponding to regions D5 and D6 are display images magnified 40 times. The display control device 13 acquires and displays tile images of the classes corresponding to these magnifications out of the tile image group of a hierarchical structure stored in the server 12. For example, the class of tile images corresponding to regions D1 and D2 is higher than the class of tile images corresponding to region D3 (that is, a class nearer to tile image T1 illustrated in FIG. 6).

While a pathological image is viewed in the above manner, the display control device 13 acquires viewing information at a predetermined sampling period. Specifically, the display control device 13 acquires the center coordinates and the display magnification of the viewed pathological image at predetermined timings, and stores the acquired viewing information in the storage section of the server 12.

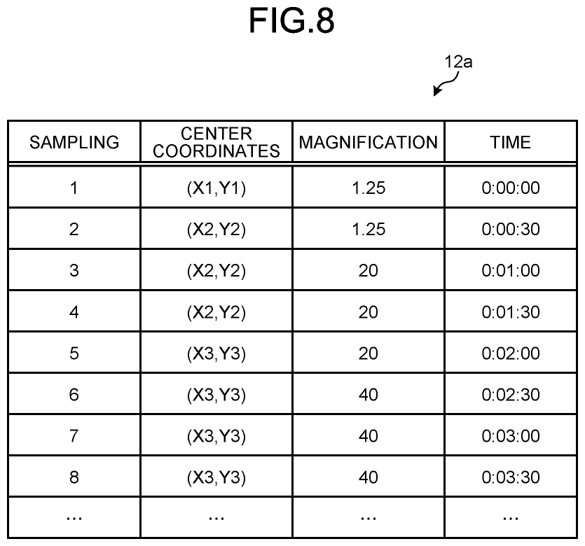

This point will now be described using FIG. 8. FIG. 8 is a diagram illustrating an example of a viewing history storage section 12a included in the server 12. As illustrated in FIG. 8, the viewing history storage section 12a stores information such as “sampling”, “center coordinates”, “magnification”, and “time”. The “sampling” indicates the order of timing of storing viewing information. The “center coordinates” indicate position information of the viewed pathological image. In this example, the center coordinates are coordinates indicated by the center position of the viewed pathological image, and correspond to coordinates of the coordinate system of the tile image group of the lowest layer. The “magnification” indicates the display magnification of the viewed pathological image. The “time” indicates the elapsed time from the start of viewing. The example of FIG. 8 shows that the sampling period is 30 seconds. That is, the display control device 13 saves viewing information in the viewing history storage section 12a every 30 seconds. However, the present invention is not limited to this example, and the sampling period may be, for example, 0.1 to 10 seconds, or may be outside this range.

In the example of FIG. 8, sampling “1” indicates viewing information of region D1 illustrated in FIG. 7, sampling “2” indicates viewing information of region D2, pieces of sampling “3” and “4” indicate viewing information of region D3, sampling “5” indicates viewing information of region D4, and pieces of sampling “6”, “7”, and “8” indicate viewing information of region D5. That is, the example of FIG. 8 shows that region D1 was viewed for about 30 seconds, region D2 was viewed for about 30 seconds, region D3 was viewed for about 60 seconds, region D4 was viewed for about 30 seconds, and region D5 was viewed for about 90 seconds. In this way, the viewing time of each region can be extracted from viewing history information.

Further, the number of times that each region was viewed can be extracted from viewing history information. For example, it is assumed that the number of times of displaying of each pixel of the displayed pathological image increases by one each time the operation of changing the display region (for example, the operation of moving the display region or the operation of changing the display size) is performed. For example, in the example illustrated in FIG. 7, if region D1 is first displayed, the number of times of displaying of each pixel included in region D1 is one. Next, if region D2 is displayed, the number of times of displaying of each pixel included in both region D1 and region D2 is two, and the number of times of displaying of each pixel not included in region D1 but included in region D2 is one. Since the display region can be identified by referring to the center coordinates and the magnification of the viewing history storage section 12a, the number of times that each pixel (which can also be referred to as each coordinate) of the pathological image is displayed can be extracted by analyzing viewing history information stored in the viewing history storage section 12a.

If the operation of changing the display position is not performed by the viewer for a predetermined period of time (for example, five minutes), the display control device 13 may suspend the processing of storing viewing information. Further, although the above example shows an example in which the viewed pathological image is stored as viewing information by means of the center coordinates and the magnification, the present invention is not limited to this example, and the viewing information may be any information as long as it can identify the region of the viewed pathological image. For example, the display control device 13 may store, as information of viewing of the pathological image, tile identification information that identifies the tile image corresponding to the viewed pathological image or information indicating the position of the tile image corresponding to the viewed pathological image. Further, although illustration is omitted in FIG. 8, information that identifies a patient, a medical record, etc. is stored in the viewing history storage section 12a. That is, the viewing history storage section 12a illustrated in FIG. 8 is stored while viewing information, and the patient, the medical record, etc. can be associated.

[2-3. Diagnostic Information]

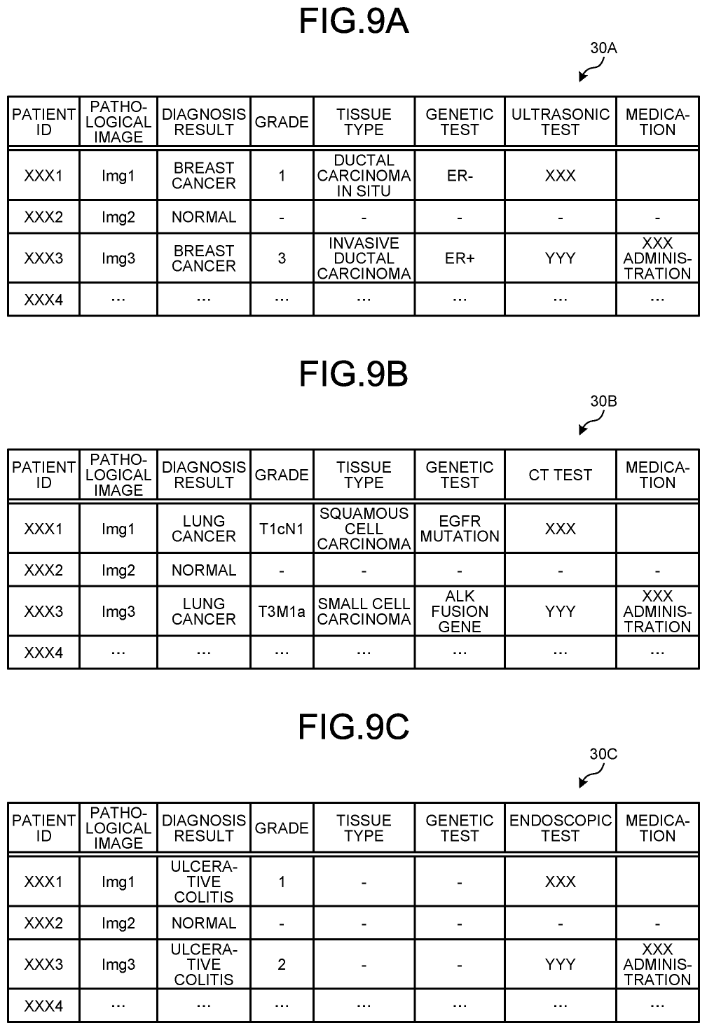

Next, diagnostic information stored in the pathology system 10 is described using FIG. 9A to FIG. 9C. FIG. 9A to FIG. 9C are diagrams illustrating diagnostic information storage sections included in the pathology system 10. FIG. 9A to FIG. 9C illustrate examples in which diagnostic information is stored in different tables for various organs to be examined. For example, FIG. 9A illustrates an example of a table that stores diagnostic information regarding a breast cancer test, FIG. 9B illustrates an example of a table that stores diagnostic information regarding a lung cancer test, and FIG. 9C illustrates an example of a table that stores diagnostic information regarding a large intestine test.

Diagnostic information storage section 30A illustrated in FIG. 9A stores information such as “patient ID”, “pathological image”, “diagnosis result”, “grade”, “tissue type”, “genetic test”, “ultrasonic test”, and “medication”. The “patient ID” indicates identification information for identifying a patient. The “pathological image” indicates a pathological image saved by a pathologist during diagnosis. In the “pathological image”, position information (center coordinates, magnification, etc.) indicating an image region to be saved with respect to the overall image may be stored instead of the image itself. The “diagnosis result” is a result of a diagnosis by a pathologist, and indicates, for example, the presence or absence of a lesion site and the type of the lesion site. The “grade” indicates the degree of progression of a disease site. The “tissue type” indicates the type of a disease site. The “genetic test” indicates a result of a genetic test. The “ultrasonic rest” indicates a result of an ultrasonic test. The medication indicates information regarding medication to a patient.

Diagnostic information storage section 30B illustrated in FIG. 9B stores information regarding a “CT test” performed in a lung cancer test instead of the “ultrasonic test” stored in diagnostic information storage section 30A illustrated in FIG. 9A. Diagnostic information storage section 30C illustrated in FIG. 9C stores information regarding an “endoscopic test” performed in a large intestine test instead of the “ultrasonic test” stored in diagnostic information storage section 30A illustrated in FIG. 9A.

In the examples of FIG. 9A to FIG. 9C, the case where “normal” is stored in the “diagnosis result” indicates that the result of the pathological diagnosis is negative, and the case where information other than “normal” is stored in the “diagnosis result” indicates that the result of the pathological diagnosis is positive. Although FIG. 9A to FIG. 9C describe cases where the items (the pathological image, the diagnosis result, the grade, the tissue type, the genetic test, the ultrasonic test, and the medication) are stored while being associated with the patient ID, it is sufficient that information regarding a diagnosis and a test be stored while being associated with the patient ID, and not all the items are necessary.

### 3. GENERATION DEVICE ACCORDING TO PRESENT EMBODIMENT

[3-1. Processing of Generation Device]

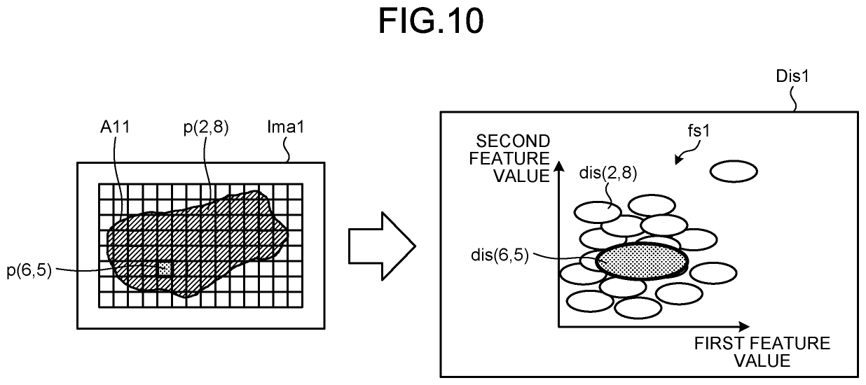

Next, an example of processing of the generation device 100 according to the present embodiment is described. FIG. 10 is a diagram for describing processing of the generation device 100 according to the present embodiment. In FIG. 10, a description is given using a pathological image Ima1. It is assumed that the pathological image Ima1 includes a cell region (observation target) A11. The generation device 100 divides the pathological image Ima1 by means of a grid or superpixels, and thus sets a plurality of partial regions. In the following description, when a partial region of the n-th row and the m-th column is indicated, it is written as partial region p(n, m), as appropriate. The plurality of partial regions are candidates for an analysis target region, a “ROI (region of interest)”.

On the other hand, the generation device 100 generates, for example, characteristic distribution information Dis1 indicating feature space fs1 formed of two axes (or one axis). For example, it is assumed that the horizontal axis of feature space fs1 is an axis corresponding to a first feature value among a plurality of kinds of feature values. It is assumed that the vertical axis of feature space fs1 is an axis corresponding to a second feature value among the plurality of kinds of feature values. It is assumed that the first feature value and the second feature value are set in advance.

The generation device 100 executes the following processing on the plurality of partial regions included in the pathological image Ima1. The generation device 100 calculates the feature values (the first feature value and the second feature value) included in the partial region, and visibly places a distribution (the range of a distribution) on feature space fs1 specified by the calculated feature values. Each of the feature values of the partial region is information having a spread in distribution, such as a maximum value and a minimum value.

For example, when the distribution of feature space fs1 specified by the feature values of partial region p(2, 8) is distribution dis(2, 8), the generation device 100 visibly places distribution dis(2, 8) in feature space fs1. Distribution dis(2, 8) is associated with partial region p(2, 8). The generation device 100 repeatedly executes the above processing on the other partial regions as well, and thus generates characteristic distribution information Dis1. In the following description, the distribution of partial region p(n, m) visibly placed on feature space fs1 is simply written as dis (n, m).

The generation device 100 displays the pathological image Ima1 divided in a plurality of partial regions and characteristic distribution information Dis1 on the display screen. Upon accepting the selection of a partial region from a user who refers to the display screen, the generation device 100 announces the distribution corresponding to the selected partial region. For example, upon accepting the selection of partial region p(6, 5) from the user, the generation device 100 highlights distribution dis(6, 5) of partial region p(6, 5).

Thus, upon accepting the selection of a partial region, the generation device 100 visually announces the distribution of the selected partial region; thereby, the user can grasp the characteristic distribution of the selected partial region. Therefore, the generation device 100 makes it possible to support the selection of a partial region having variations in characteristics. That is, while understanding the spread of the characteristic distribution of each partial region, the user can select, as an analysis target partial region (ROI), a partial region having target characteristics from the plurality of partial regions.

Although the example illustrated in FIG. 10 describes a case where the generation device 100 displays pathological image Ima1 and characteristic distribution information Dis1 separately, pathological image Ima1 and characteristic distribution information Dis1 may be displayed side by side on the same screen.

[3-2. Functional Configuration of Generation Device]

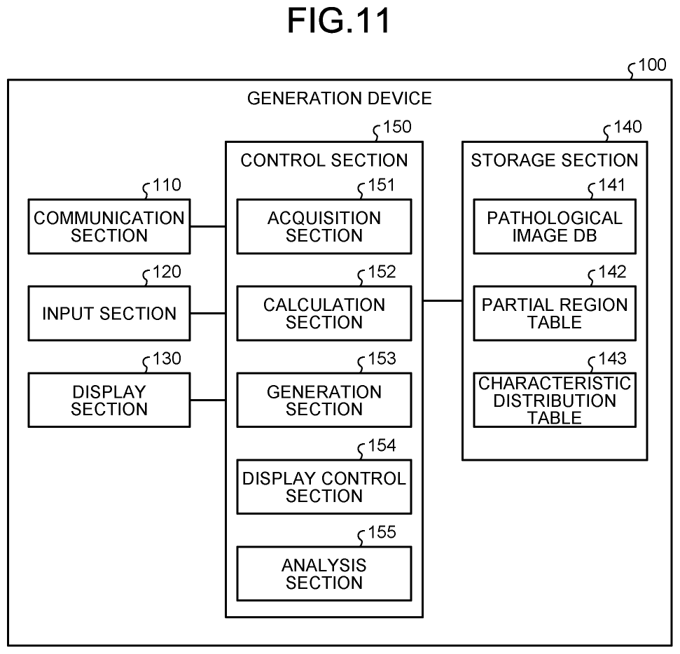

Next, an example of a functional configuration of the generation device 100 according to the present embodiment is described. FIG. 11 is a functional block diagram illustrating a configuration of a generation device according to the present embodiment. As illustrated in FIG. 11, the generation device 100 includes a communication section 110, an input section 120, a display section 130, a storage section 140, and a control section 150.

The communication section 110 is obtained by using, for example, a network interface card (NIC) or the like. The communication section 110 is connected to a not-illustrated network in a wired or wireless manner, and performs the transmission and reception of information with the pathology system 10, etc. via the network. The control section 150 described later performs the transmission and reception of information with these devices via the communication section 110.

The input section 120 is an input device through which various pieces of information are inputted to the generation device 100. The input section 120 corresponds to a keyboard, a mouse, a touch panel, or the like. The user operates the input section 120 to select a partial region, etc. on a pathological image.

The display section 130 is a display device that displays information outputted from the control section 150. The display section 130 corresponds to a liquid crystal display, an organic EL (electro-luminescence) display, a touch panel, or the like. For example, the display section 130 displays a pathological image, characteristic distribution information, etc. described in FIG. 10.

The storage section 140 includes a pathological image DB (data base) 141, a partial region table 142, and a characteristic distribution table 143. The storage section 140 is obtained by using, for example, a semiconductor memory element such as a RAM (random access memory) or a flash memory, or a storage device such as a hard disk or an optical disk.

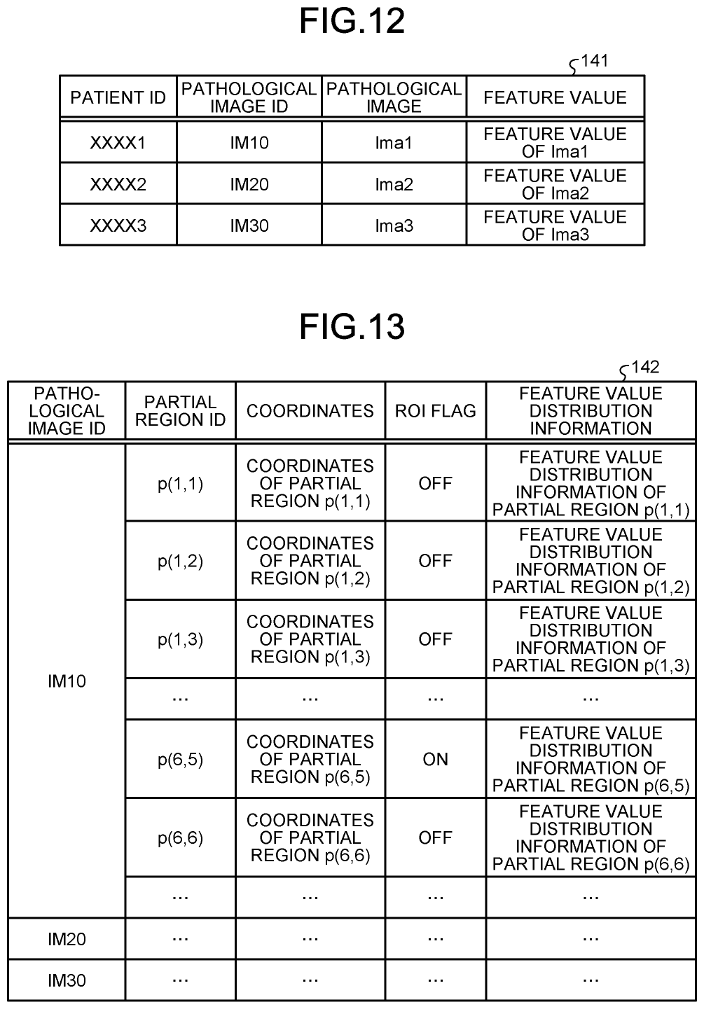

The pathological image DB 141 is a database that stores a plurality of pathological images. FIG. 12 is a diagram illustrating an example of a data structure of the pathological image DB 141. As illustrated in FIG. 12, the pathological image DB 141 includes “patient ID”, “pathological image ID”, “pathological image”, and “feature values”. The patient ID is information that uniquely identifies a patient. The pathological image ID is information that uniquely identifies a pathological image. The pathological image indicates a pathological image (image data) saved by a pathologist during diagnosis. The pathological image is transmitted from the server 12. The pathological image corresponds to a whole slide image (WSI) or the like.

The feature value is a feature value obtained from the entirety of one corresponding pathological image. For example, the feature value includes information of a result of extraction of each cell present in a pathological image, an image element, and the distance between each cell and a specific tumor region. The result of extraction of each cell includes “the size, shape, and staining intensity regarding the cell nucleus”, “the size, shape, staining intensity, cell density regarding the cell membrane”, etc. The image element includes a CNN (convolution neural network) feature value, a color, a luminance value, frequency characteristics, etc.

The pathological image DB 141 may hold information such as “diagnosis result”, “grade”, “tissue type”, “genetic test”, “ultrasonic test”, and “medication” described in FIG. 9A to FIG. 9C in addition to the patient ID, the pathological image, and the feature value. The feature value may be calculated by a calculation section 152 described later on the basis of the pathological image, or may be acquired from the server 12.

The partial region table 142 is a table that holds information regarding a plurality of partial regions obtained by dividing a pathological image. FIG. 13 is a diagram illustrating an example of a data structure of the partial region table 142. As illustrated in FIG. 13, the partial region table 142 includes “pathological image ID”, “partial region ID”, “coordinates”, “ROI flag”, and “feature value distribution information”.

The pathological image ID is information that uniquely identifies a pathological image. A plurality of partial regions are set for each pathological image. The partial region ID is information that identifies each partial region in the pathological image. The coordinates indicate the coordinates of the partial region. For example, the coordinates of the partial region are specified by the coordinates of the upper left corner and the coordinates of the lower right corner of the partial region. The ROI flag indicates whether the corresponding partial region (the partial region specified by the partial region ID) is registered as a ROI or not. For example, in the case where the corresponding partial region is registered as a ROI, the ROI flag corresponding to the partial region ID is “ON”. In the case where the corresponding partial region is not registered as a ROI, the ROI flag corresponding to the partial region ID is “OFF”. In the following description, the partial region of partial region ID “p(n, m)” is simply written as partial region p(n, m).

The feature value distribution information is information of a distribution of feature values in one corresponding partial region. The feature value distribution information includes information that defines a representative point and information that defines the spread of a distribution. The information that defines a representative point includes the average value, median value, mode value, or the like of the feature values. The information that defines the spread of a distribution includes a histogram, a variance, a standard deviation, a maximum value and a minimum value, a quartile, or the like.

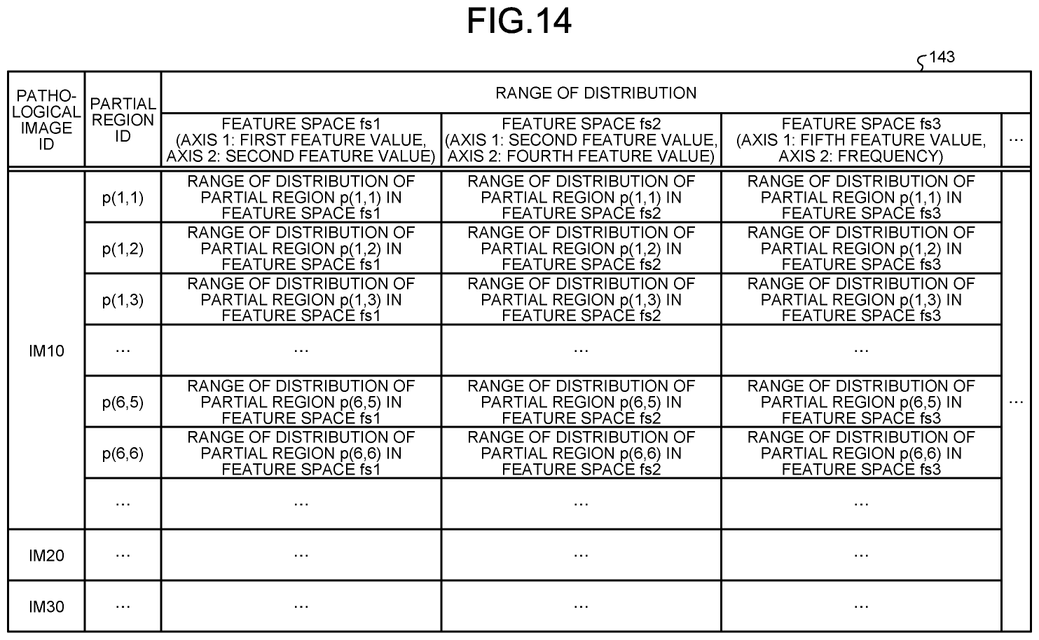

The characteristic distribution table 143 is a table that holds information regarding a distribution of a partial region in a feature space. FIG. 14 is a diagram illustrating an example of a data structure of a characteristic distribution table. As illustrated in FIG. 14, the characteristic distribution table 143 associates a pathological image ID, a partial region ID, and the range of a distribution. The pathological image ID is information that uniquely identifies a pathological image. The partial region ID is information that identifies a partial region in a pathological image.

The range of a distribution is information indicating the range of a distribution of a partial region in a feature space. For the feature space, there may be different feature spaces depending on the kind of feature value assigned to an axis of the feature space. The distribution of a partial region is set for each of different feature spaces. In the example illustrated in FIG. 14, the ranges of distributions of a partial region corresponding to feature spaces fs1, fs2, fs3, etc. are set. For example, feature space fs1 is a feature space having axes of a first feature value and a second feature value. Feature space fs2 is a feature space having axes of a second feature value and a fourth feature value. Feature space fs3 is a feature space (histogram) having an axis of a fifth feature value. Although FIG. 14 illustrates the ranges of distributions of feature spaces fs1 to fs3, the characteristic distribution table 143 may have the range of a distribution of a feature space having an axis of another kind of feature value.

The description returns to FIG. 11. The control section 150 includes an acquisition section 151, a calculation section 152, a generation section 153, a display control section 154, and an analysis section 155. The control section 150 is operated by, for example, a process in which a CPU (central processing unit) or an MPU (micro processing unit) executes a program (an example of an analysis program) stored in the generation device 100 by using a RAM (random access memory) or the like as a work area. The control section 150 may be executed by, for example, an integrated circuit such as an ASIC (application-specific integrated circuit) or an FPGA (field programmable gate array).

The acquisition section 151 is a processing section that sends a request to acquire a pathological image to the server 12 and acquires the pathological image from the server 12. The acquisition section 151 registers the acquired pathological image in the pathological image DB 141. It is assumed that a pathological image ID and a patient ID are given to the pathological image. In the case where information of a feature value of the pathological image is attached to the pathological image in addition to the patient ID, the acquisition section 151 registers the feature value of the pathological image in the pathological image DB 141. Further, information such as “diagnosis result”, “grade”, “tissue type”, “genetic test”, “ultrasonic test”, and “medication” described in FIG. 9A to FIG. 9C may be attached to the pathological image.

The calculation section 152 is a processing section that divides a pathological image into a plurality of partial regions and calculates feature value distribution information of each partial region.

An example of processing by the calculation section 152 of dividing a pathological image into a plurality of partial regions will now be described. The calculation section 152 acquires a pathological image from the pathological image DB 141, and divides the pathological image into a plurality of partial regions by means of a grid, superpixels, or the like set in advance. The calculation section 152 assigns a partial region ID to each partial region, and registers the pathological image ID, the partial region ID, and the coordinates in the partial region table 142 while associating them together. The calculation section 152 sets the ROI flag corresponding to the partial region ID to the initial value, “OFF”.

An example of processing by the calculation section 152 of calculating feature value distribution information of each partial region will now be described. The calculation section 152 acquires feature values corresponding to partial region p(n, m) from among the feature values of the pathological image registered in the pathological image DB 141. The feature values corresponding to partial region p(n, m) include feature values of a cell (the size, shape, and staining intensity of the nucleus), the size of the cell membrane, the staining intensity of the cell membrane, and the cell density. The feature values corresponding to partial region p(n, m) include information of the distance between a cell included in partial region p(n, m) (or partial region p(n, m)) and a specific region (a tumor region given separately). The feature values corresponding to partial region p(n, m) include an image feature value (a CNN feature value, color information, frequency characteristics, etc.).

The calculation section 152 calculates feature value distribution information on the basis of the feature values corresponding to partial region p(n, m). For example, the calculation section 152 calculates, for each kind of feature value, information that defines a representative point and information that defines the spread of a distribution. The information that defines a representative point includes the average value, median value, mode value, or the like of the feature values. The information that defines the spread of a distribution includes a histogram, a variance, a standard deviation, a maximum value and a minimum value, a quartile, or the like. The calculation section 152 registers the feature value distribution information of partial region p(n, m) in the partial region table 142.

The calculation section 152 generates the partial region table 142 by repeatedly executing the above processing for each partial region included in the pathological image. Further, the calculation section 152 may repeatedly execute the above processing for a plurality of pathological images registered in the pathological image DB 141.

The generation section 153 is a processing section that generates the characteristic distribution table 143 on the basis of the partial region table 142. Hereinbelow, an example of processing of the generation section 153 is described.

The generation section 153 acquires feature value distribution information corresponding to partial region p(n, m) from the partial region table 142. The generation section 153 acquires information of a feature space set in advance. Herein, a description is given using feature space fs1. Feature space fs1 is a feature space having two axes of a first feature value and a second feature value.

The generation section 153 acquires “information that defines the spread of a distribution” corresponding to the first feature value and “information that defines the spread of a distribution” corresponding to the second feature value from feature value distribution information corresponding to partial region p(n, m). The generation section specifies the range of a distribution of partial region p(n, m) on feature space fs1 on the basis of the acquired information, and registers the result in the characteristic distribution table 143. The generation section 153 specifies the range of a distribution in another feature space for partial region p(n, m), and registers the result in the characteristic distribution table 143.

The generation section 153 generates the characteristic distribution table 143 by repeatedly executing the above processing for each partial region included in the pathological image. Further, the generation section 153 may repeatedly execute the above processing for each partial region of a plurality of pathological images.

The display control section 154 is a processing section that generates screen information including a pathological image and characteristic distribution information on the basis of the pathological image DB 141, the partial region table 142, and the characteristic distribution table 143, and causes the display section 130 to display the screen information. Hereinbelow, various pieces of processing executed by the display control section 154 are described.

Processing (1) executed by the display control section 154 will now be described. Processing (1) executed by the display control section 154 will now be described using FIG. 10. The display control section 154 causes the display section 130 to display pathological image Ima1 divided in a plurality of partial regions. The display control section 154 sets a plurality of partial regions for pathological image Ima1 on the basis of the partial region table 142. The user refers to pathological image Ima1 and operates the input section 120 to select any partial region.

Upon accepting the selection of any partial region among the plurality of partial regions of pathological image Ima1, the display control section 154 causes the display section 130 to display characteristic distribution information Dis1. For example, the display control section 154 specifies the range of a distribution of each partial region corresponding to feature space fs1 on the basis of the characteristic distribution table 143, and visually arranges the distributions of the partial regions in feature space fs1; thereby, generates characteristic distribution information Dis1. The display control section 154 highlights the distribution corresponding to the partial region selected by the user among the plurality of distributions arranged in characteristic distribution information Dis1.

For example, upon accepting the selection of partial region p(6, 5) from the user, the display control section 154 highlights distribution dis(6, 5) of partial region p(6, 5). Although illustration is omitted, the display control section 154 may enlarge and display the selected partial region p(6, 5).

Upon accepting a registration request for the selected partial region from the user, the display control section 154 refers to the partial region table 142 and sets the ROI flag corresponding to the partial region for which a registration request has been accepted to “ON”.

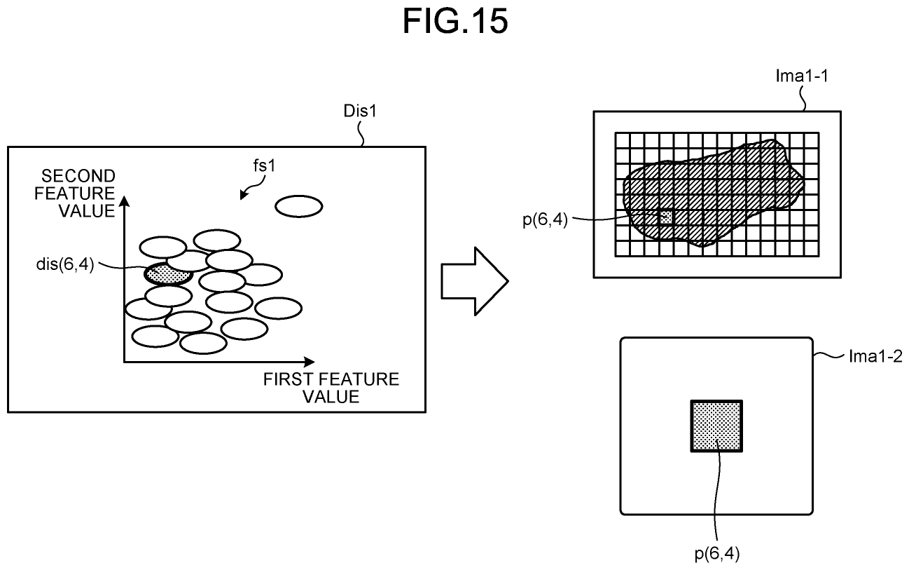

Processing (2) executed by the display control section 154 will now be described. FIG. 15 is a diagram for describing processing (2) executed by the display control section 154. The display control section 154 generates characteristic distribution information Dis1 on the basis of the characteristic distribution table 143, and causes the display section 130 to display characteristic distribution information Dis1. The user refers to characteristic distribution information Dis1 and operates the input section 120 to select any distribution.

Upon accepting the selection of a distribution of characteristic distribution information Dis1, the display control section 154 causes the display section 130 to display pathological image Ima1-1. The display control section 154 highlights the partial region corresponding to the distribution selected by the user among the plurality of partial regions of pathological image Ima1-1.

For example, when distribution dis(6, 4) of characteristic distribution information Dis1 is selected by the user, the display control section 154 highlights partial region p(6, 4) of pathological image Ima1-1. The display control section 154 may display image Ima1-2 in which partial region p(6, 4) is enlarged.

Upon accepting a registration request for partial region p(6, 4) from the user, the display control section 154 refers to the partial region table 142 and sets the ROI flag corresponding to partial region p(6, 4) for which a registration request has been accepted to “ON”.

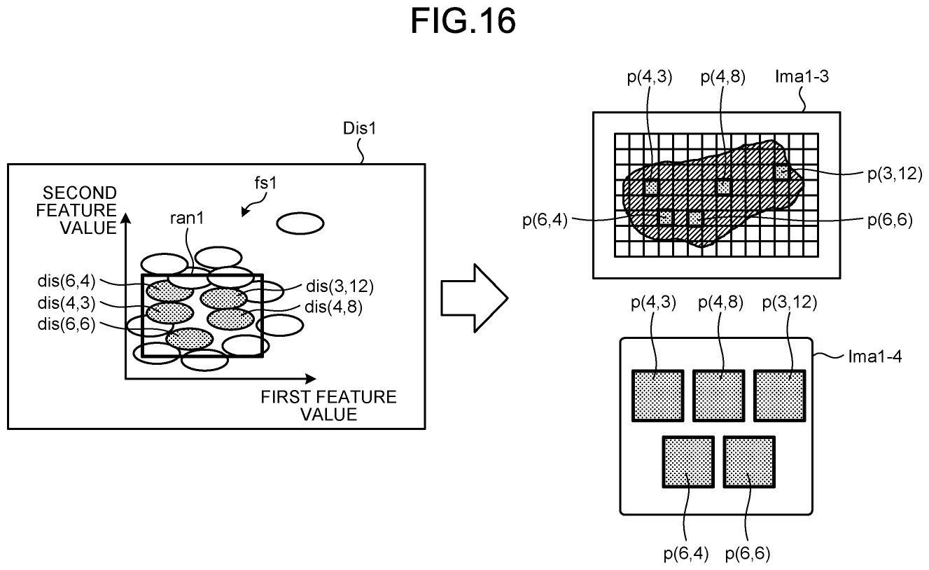

Processing (3) executed by the display control section 154 will now be described. FIG. 16 is a diagram for describing processing (3) executed by the display control section 154. The display control section 154 generates characteristic distribution information Dis1 on the basis of the characteristic distribution table 143, and causes the display section 130 to display characteristic distribution information Dis1. The user refers to characteristic distribution information Dis1 and operates the input section 120 to select any distribution (a plurality of distributions). The user may select a plurality of distributions by specifying a range on characteristic distribution information Dis1.

Upon accepting the selection of distributions of characteristic distribution information Dis1, the display control section 154 causes the display section 130 to display pathological image Ima1-3. The display control section 154 highlights the partial regions corresponding to the distributions selected by the user among the plurality of partial regions of pathological image Ima1-3.

For example, the display control section 154 assumes that range ran1 is specified by the user and distributions dis(4, 3), dis(4, 8), dis(3, 12), dis(6, 4), and dis(6, 6) of characteristic distribution information Dis1 are selected. In this case, the display control section 154 highlights partial regions p(4, 3), p(4, 8), p(3, 12), p(6, 4), and p(6, 6) of pathological image Ima1-3. The display control section 154 may display image Ima1-4 in which partial regions p(4, 3), p(4, 8), p(3, 12), p(6, 4), and p(6, 6) are enlarged.

Upon accepting a registration request for partial regions p(4, 3), p(4, 8), p(3, 12), p(6, 4), and p(6, 6) from the user, the display control section 154 refers to the partial region table 142 and sets the ROI flags corresponding to the partial regions for which a registration request has been accepted to “ON”.

In the case where the number of partial regions to be registered as the ROI has been specified and the number of distributions of partial regions included in range ran1 is beyond the specified number, the display control section 154 may randomly select distributions of partial regions of range ran1 so as to hold the specified number. Further, the display control section 154 may select the specified number so that the distributions of partial regions are scattered. By providing such a function, for example, partial regions that have a high degree of irregularity such as a large area and are close to a tumor margin can be presented.

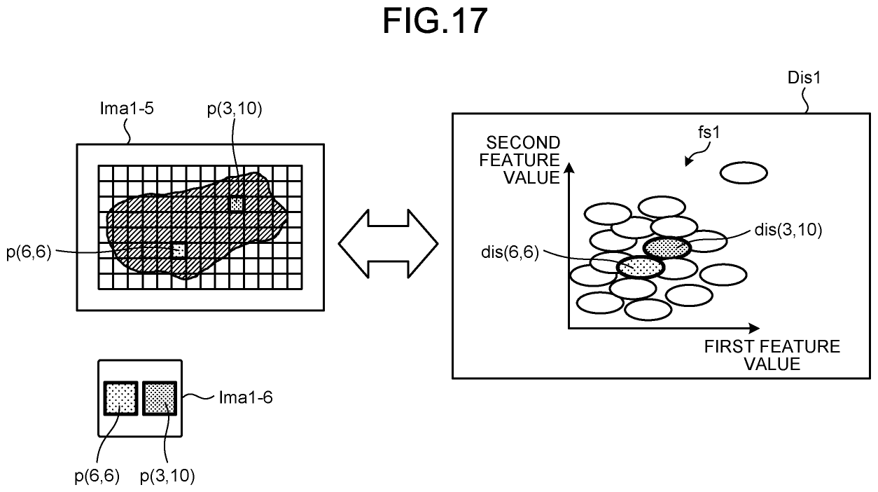

Processing (4) executed by the display control section 154 will now be described. FIG. 17 is a diagram for describing processing (4) executed by the display control section 154. The display control section 154 causes the display section 130 to display pathological image Ima1-5 divided in a plurality of partial regions. The user refers to pathological image Ima1-5 and operates the input section 120 to select any partial region.

Upon accepting the selection of any partial region among the plurality of partial regions of pathological image Ima1-5, the display control section 154 causes the display section 130 to display characteristic distribution information Dis1. The display control section 154 highlights the distribution corresponding to the partial region selected by the user among the plurality of distributions arranged in characteristic distribution information Dis1.

For example, upon accepting the selection of partial region p(6, 6) from the user, the display control section 154 highlights distribution dis(6, 6) of partial region p(6, 6). Upon accepting a registration request for partial region p(6, 6) from the user, the display control section 154 sets the ROI flag corresponding to partial region p(6, 6) of the partial region table 142 to “ON”.

Upon accepting a request to display pathological image Ima1-5 from the user who operates the input section 120, the display control section 154 causes the display section 130 to display pathological image Ima1-5. The display control section 154 refers to the partial region table 142 and highlights partial region p(6, 6), where the ROI flag is “ON”.

Upon accepting the selection of any partial region among the plurality of partial regions of pathological image Ima1-5, the display control section 154 causes the display section 130 to display characteristic distribution information Dis1. The display control section 154 highlights the distribution corresponding to the partial region selected by the user among the plurality of distributions arranged in characteristic distribution information Dis1. Further, the display control section 154 highlights the distribution corresponding to a partial region where the ROI flag is “ON”.

For example, upon accepting the selection of partial region p(3, 10) from the user, the display control section 154 highlights distribution dis(3, 10) of partial region p(3, 10). Further, the display control section 154 highlights distribution dis(6, 6) corresponding to partial region p(6, 6), where the ROI flag is “ON”. The display control section 154 may display image Ima1-6 in which partial regions p(6, 6) and p(3, 10) are enlarged.

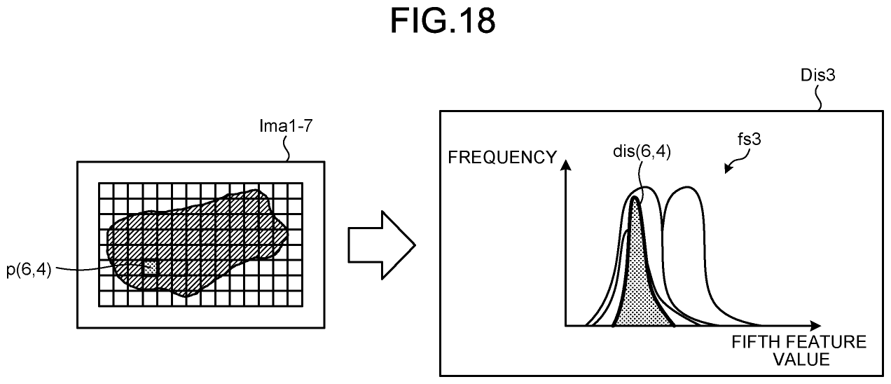

Processing (5) executed by the display control section 154 will now be described. FIG. 18 is a diagram for describing processing (5) executed by the display control section 154. The display control section 154 causes the display section 130 to display pathological image Ima1-7 divided in a plurality of partial regions. The user refers to pathological image Ima1-7 and operates the input section 120 to select any partial region.

Upon accepting the selection of any partial region among the plurality of partial regions of pathological image Ima1-7, the display control section 154 causes the display section 130 to display characteristic distribution information Dis3. Characteristic distribution information Dis3 indicates feature space fs3. For example, it is assumed that the horizontal axis of feature space fs3 is an axis corresponding to a fifth feature value among a plurality of kinds of feature values. It is assumed that the vertical axis of feature space fs3 is an axis corresponding to frequency. Feature space fs3 corresponds to a histogram.

The display control section 154 highlights the distribution corresponding to the partial region selected by the user among the plurality of distributions arranged in characteristic distribution information Dis3. For example, upon accepting the selection of partial region p(6, 6) from the user, the display control section 154 highlights distribution dis(6, 6) of partial region p(6, 6). Upon accepting a registration request for partial region p(6, 6) from the user, the display control section 154 sets the ROI flag corresponding to partial region p(6, 6) of the partial region table 142 to “ON”.

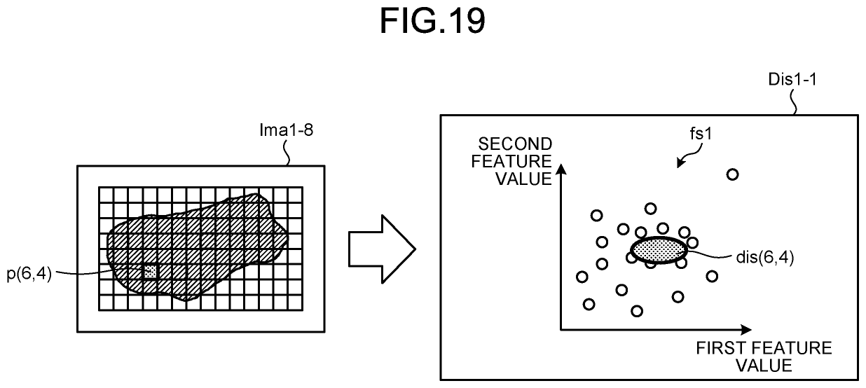

Processing (6) executed by the display control section 154 will now be described. FIG. 19 is a diagram for describing processing (6) executed by the display control section 154. The display control section 154 causes the display section 130 to display pathological image Ima1-8 divided in a plurality of partial regions. The user refers to pathological image Ima1-8 and operates the input section 120 to select any partial region.

Upon accepting the selection of any partial region among the plurality of partial regions of pathological image Ima1-8, the display control section 154 causes the display section 130 to display characteristic distribution information Dis1-1. The display control section 154 highlights the distribution corresponding to the partial region selected by the user among the plurality of distributions arranged in characteristic distribution information Dis1-1.

For example, upon accepting the selection of partial region p(6, 4) from the user, the display control section 154 highlights distribution dis(6, 4) of partial region p(6, 4). Upon accepting a registration request for partial region p(6, 4) from the user, the display control section 154 sets the ROI flag corresponding to partial region p(6, 4) of the partial region table 142 to “ON”.

When causing the display section 130 to display characteristic distribution information Dis1-1, the display control section 154 causes the distributions other than the distribution of the selected partial region to be displayed by means of representative points. The representative point corresponds to an average value, a median value, or the like. In the example illustrated in FIG. 19, the display control section 154 causes the distributions other than distribution dis(6, 4) of partial region p(6, 4) to be displayed by means of representative points.

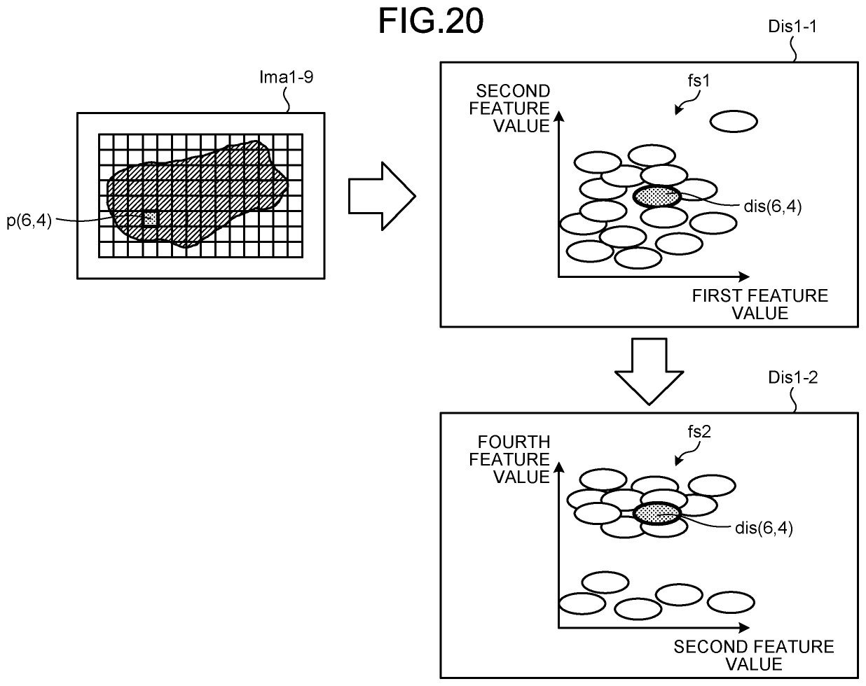

Processing (7) executed by the display control section 154 will now be described. FIG. 20 is a diagram for describing processing (7) executed by the display control section 154. The display control section 154 causes the display section 130 to display pathological image Ima1-9 divided in a plurality of partial regions. The user refers to pathological image Ima1-9 and operates the input section 120 to select any partial region.

Upon accepting the selection of any partial region among the plurality of partial regions of pathological image Ima1-9, the display control section 154 causes the display section 130 to display characteristic distribution information Dis1-1 of feature space fs1. The display control section 154 highlights the distribution corresponding to the partial region selected by the user among the plurality of distributions arranged in characteristic distribution information Dis1-1.

For example, upon accepting the selection of partial region p(6, 4) from the user, the display control section 154 highlights distribution dis(6, 4) of partial region p(6, 4) in characteristic distribution information Dis1-1.

Here, upon accepting a request to change the feature space from the user, the display control section 154 displays characteristic distribution information indicating the feature space after change. For example, when feature space fs2 is selected by the user, the display control section 154 displays characteristic distribution information Dis1-2. The display control section 154 highlights distribution dis(6, 4) of partial region p(6, 4) in characteristic distribution information Dis1-2.

Upon accepting a registration request for partial region p(6, 4) from the user, the display control section 154 sets the ROI flag corresponding to partial region p(6, 4) of the partial region table 142 to “ON”.

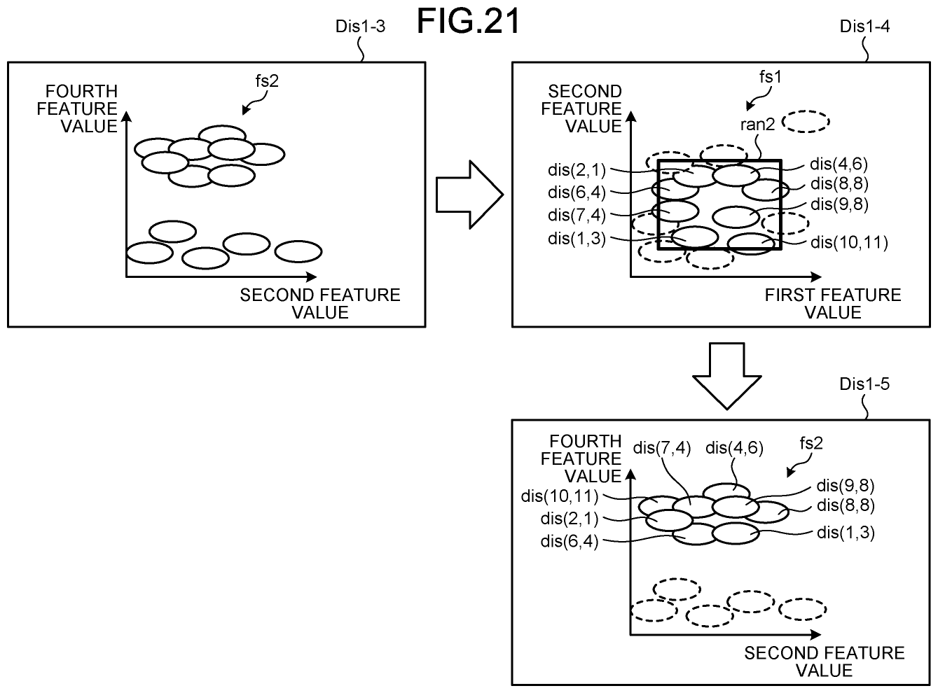

Processing (8) executed by the display control section 154 will now be described. FIG. 21 is a diagram for describing processing (8) executed by the display control section 154. When feature space fs1 is specified by the user, the display control section 154 generates characteristic distribution information Dis1-3 on the basis of the characteristic distribution table 143, and causes the display section 130 to display characteristic distribution information Dis1-3.

Subsequently, when feature space fs2 is specified by the user, the display control section 154 generates characteristic distribution information Dis1-4 on the basis of the characteristic distribution table 143, and causes the display section 130 to display characteristic distribution information Dis1-4.

For example, when by the user, range ran2 is specified, distributions dis(2, 1), dis(6, 4), dis(7, 4), dis(1, 3), dis(9, 8), dis(10, 11), dis(8, 8), and dis(4, 6) of characteristic distribution information Dis1-4 are selected, and then feature space fs1 is specified, the display control section 154 causes the display section 130 to display characteristic distribution information Dis1-5.

The display control section 154 causes distributions of feature space fs2 that correspond to the distributions of feature space fs1 selected in characteristic distribution information Dis1-4 to be displayed in characteristic distribution information Dis1-5. In the example illustrated in FIG. 21, distributions dis(2, 1), dis(6, 4), dis(7, 4), dis(1, 3), dis(9, 8), dis(10, 11), dis(8, 8), and dis(4, 6) of characteristic distribution information 1-5 are displayed, and the other distributions are not displayed. That is, when an effective region is specified in a certain feature space fs1, distributions corresponding to the effective region are displayed in feature space fs2.

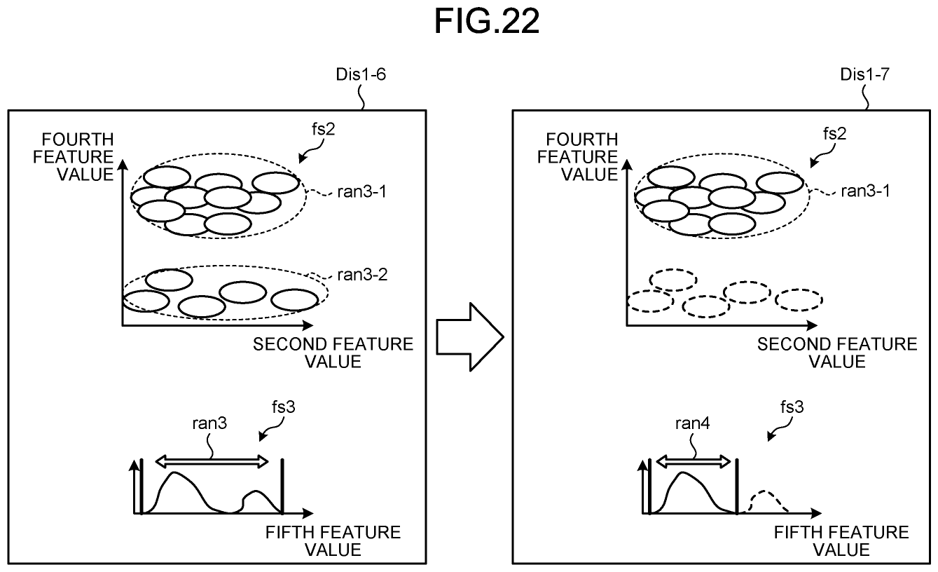

Processing (9) executed by the display control section 154 will now be described. FIG. 22 is a diagram for describing processing (9) executed by the display control section 154. When feature spaces fs2 and fs3 are specified by the user, the display control section 154 generates characteristic distribution information Dis1-6 on the basis of the characteristic distribution table 143, and causes the display section 130 to display characteristic distribution information Dis1-6. For example, it is assumed that the distributions of the partial regions included in range ran3 of feature space fs3 correspond to the distributions of the partial regions included in ranges ran3-1 and ran3-2 of feature space fs2.

When the range of feature space fs3 is changed from ran3 to ran4 by the user, the display control section 154 generates characteristic distribution information Dis1-7, and causes the display section 130 to display characteristic distribution information Dis1-7. For example, when the distributions of the partial regions included in range ran4 of feature space fs are the distributions of the partial regions included in range ran3-1 of feature space fs2, the display control section 154 causes only the distributions of the partial regions included in range ran3-1 of feature space fs2 to be displayed. That is, when a range is specified in a certain feature space fs3, distributions effective for the range are displayed in feature space fs2.

The description returns to FIG. 11. The analysis section 155 refers to the partial region table 142 and uses a predetermined analysis procedure to perform analysis on a partial region where the ROI flag is turned to “ON”, and outputs a final analysis result to the display section 130. The user may adjust parameters used for analysis processing while referring to the analysis result.

### 4. PROCESSING PROCEDURE

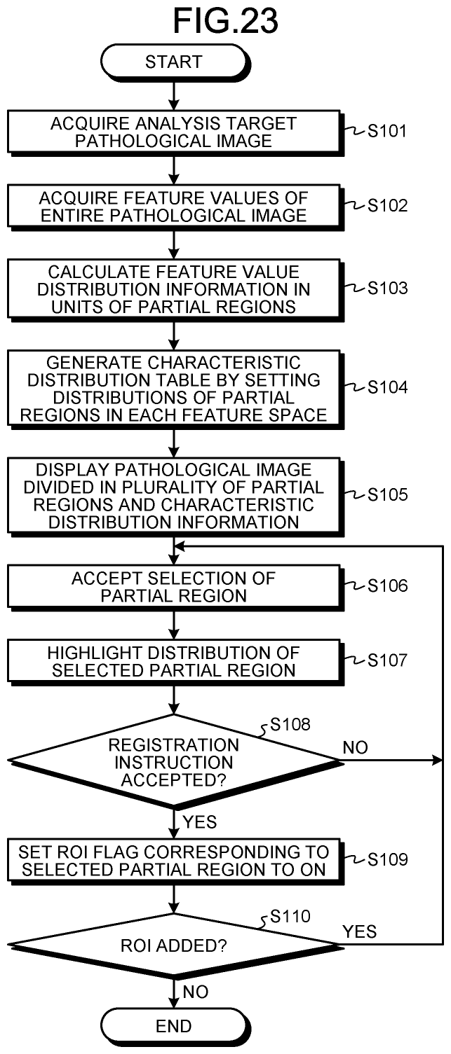

Next, a processing procedure of the generation device 100 according to the present embodiment is described using FIG. 23. FIG. 23 is a flowchart illustrating a processing procedure of a generation processing device according to the present embodiment. The acquisition section 151 of the generation device 100 acquires an analysis target pathological image (Step S101). The acquisition section 151 acquires feature values of the entire pathological image (Step S102).

The calculation section 152 of the generation device 100 calculates feature value distribution information in units of partial regions (Step S103). The generation section 153 generates the characteristic distribution table 143 by setting distributions of partial spaces in each of feature spaces (Step S104).

The display control section 154 of the generation device 100 displays a pathological image divided in a plurality of partial regions and characteristic distribution information (Step S105). The display control section 154 accepts the selection of a partial region (Step S106). The display control section 154 highlights the distribution of the selected partial region (Step S107).

In the case where the display control section 154 does not accept an instruction to register the selected partial region (Step S108: No), the display control section 154 proceeds to Step S106. On the other hand, in the case where the display control section 154 has accepted a request to register the selected partial region (Step S108: Yes), the display control section 154 proceeds to Step S109.

The display control section 154 sets the ROI flag corresponding to the selected partial region to ON (Step S109). In the case where a ROI is to be added (Step S110: Yes), the generation device 100 proceeds to Step S106. On the other hand, in the case where a ROI is not to be added (Step S110), the generation device 100 ends the processing.

### 5. EFFECTS OF GENERATION DEVICE ACCORDING TO PRESENT EMBODIMENT

In the generation device 100 according to the present embodiment, the generation section 153, on the basis of feature value distribution information of partial regions set in a pathological image, generates characteristic distribution information in which distributions of the partial regions on a feature space specified by feature values included in the partial regions are visibly arranged in the feature space while being associated with the partial regions. Thereby, the selection of a partial region having variations in characteristics can be supported.

Further, when executing parameter adjustment of analysis processing by using a ROI, adjustment in a ROI having sufficient variations in characteristics becomes possible by supporting the selection of a partial region having variations in characteristics. Thereby, the risk that, when an adjustment result is applied to another analysis target region, analysis processing will not work as intended can be reduced.

The generation device 100, on the basis of feature values of a plurality of partial regions set in an image regarding pathology, generates characteristic distribution information by arranging distributions of the plurality of partial regions in the feature space. Thereby, the selection of a plurality of partial regions having variations in characteristics can be supported.

The generation device 100 displays a pathological image in which a plurality of partial regions are set and characteristic distribution information, and when a partial region is selected, displays the distribution of characteristic distribution information corresponding to the selected partial region. Thereby, the distribution of the selected partial region in the pathological image can be easily grasped.

The generation device 100 acquires feature values corresponding to a pathological image, and calculates feature values of partial regions on the basis of the feature values corresponding to the pathological image. Thereby, a distribution of a partial region in a feature space can be specified.

The generation device 100 generates characteristic distribution information on the basis of some kinds of feature values among a plurality of kinds of feature values. For example, the generation device 100 generates characteristic distribution information on the basis of a kind of a first combination of feature values among a plurality of kinds of feature values, and generates another piece of characteristic distribution information on the basis of a kind of a second combination of feature values. Thereby, it becomes possible to display characteristic distribution information corresponding to a feature value aimed at by the user among the plurality of kinds of feature values.

In the case where the generation device 100 has accepted a registration request for a partial region, the generation device 100 saves information of a registered partial region indicating the partial region for which a registration request has been accepted, and further displays a distribution of characteristic distribution information corresponding to the registered partial region. Thereby, a distribution of a partial region (ROI) already selected by the user can be announced.

The generation device 100 displays each distribution of characteristic distribution information by means of a representative value of the feature values, and when a partial region is selected, displays the distribution of the selected partial region by means of a distribution having a spread specified by the feature values of the partial region. Thereby, the range of the distribution of the partial region can be easily viewed.

### 6. HARDWARE CONFIGURATION

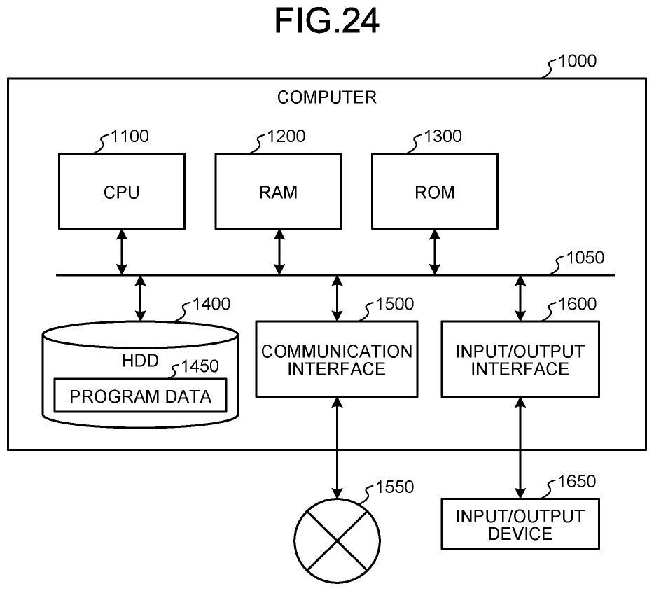

The generation device according to each embodiment described above is obtained by using, for example, a computer 1000 having a configuration like that illustrated in FIG. 24. Hereinbelow, a description is given using, as an example, the generation device 100 according to the present embodiment. FIG. 24 is a hardware configuration diagram illustrating an example of a computer 1000 that implements functions of a generation device. The computer 1000 includes a CPU 1100, a RAM 1200, a ROM (read-only memory) 1300, an HDD (hard disk drive) 1400, a communication interface 1500, and an input/output interface 1600. Each section of the computer 1000 is connected by a bus 1050.

The CPU 1100 operates on the basis of a program stored in the ROM 1300 or the HDD 1400, and controls each section. For example, the CPU 1100 develops a program stored in the ROM 1300 or the HDD 1400 onto the RAM 1200, and executes processing corresponding to various programs.

The ROM 1300 stores a boot program such as a BIOS (basic input output system) that is executed by the CPU 1100 when the computer 1000 is activated, a program depending on the hardware of the computer 1000, etc.

The HDD 1400 is a computer-readable recording medium that non-temporarily records a program to be executed by the CPU 1100, data to be used by the program, etc. Specifically, the HDD 1400 is a recording medium that records an information processing program according to the present disclosure that is an example of program data 1450.

The communication interface 1500 is an interface for the computer 1000 to connect to an external network 1550 (for example, the Internet). For example, the CPU 1100 receives data from another device or transmits data generated by the CPU 1100 to another device via the communication interface 1500.

The input/output interface 1600 is an interface for connecting an input/output device 1650 and the computer 1000. For example, the CPU 1100 receives data from an input device such as a keyboard and a mouse via the input/output interface 1600. Further, the CPU 1100 transmits data to an output device such as a display, a speaker, or a printer via the input/output interface 1600. The input/output interface 1600 may function as a media interface that reads a program, etc. recorded in a predetermined recording medium (medium). The medium is, for example, an optical recording medium such as a DVD (digital versatile disc) or a PD (phase change rewritable disk), a magneto-optical recording medium such as an MO (magneto-optical disk), a tape medium, a magnetic recording medium, a semiconductor memory, or the like.

For example, in the case where the computer 1000 functions as the generation device 100 according to the embodiment, the CPU 1100 of the computer 1000 executes a generation program loaded on the RAM 1200 to implement the functions of the acquisition section 151, the calculation section 152, the generation section 153, the display control section 154, the analysis section 155, etc. The HDD 1400 stores a generation program, etc. according to the present disclosure. Although the CPU 1100 reads the program data 1450 from the HDD 1400 and executes the program data, these programs may be acquired from another device via the external network 1550, as another example.

### 7. CONCLUSIONS

A generation device includes a generation section. The generation section, on the basis of feature values of partial regions set in an image regarding pathology, generates distribution information in which distributions of the partial regions on a feature space specified by feature values included in the partial regions are visibly arranged in the feature space while being associated with the partial regions. Thereby, the selection of a partial region having variations in characteristics can be supported.

The generation section generates the distribution information by, on the basis of feature values of a plurality of partial regions set in the image regarding pathology, arranging distributions of the plurality of partial regions in the feature space. Thereby, the selection of a plurality of partial regions having variations in characteristics can be supported.

The generation device further includes a display control section that displays an image regarding pathology in which the plurality of partial regions are set and the distribution information. When the partial region is selected, the display control section displays a distribution of the distribution information corresponding to the selected partial region. Thereby, the distribution of the selected partial region in the pathological image can be easily grasped.

The generation device further includes: an acquisition section that acquires a feature value for the image regarding pathology; and a calculation section that calculates a feature value of the partial region on the basis of a feature value acquired by the acquisition section. Thereby, a distribution of a partial region in a feature space can be specified.

The partial region has a plurality of kinds of feature values, and the generation section generates the distribution information on the basis of some kinds of feature values among the plurality of kinds of feature values. The generation section generates a first piece of distribution information on the basis of a kind of a first combination of feature values among the plurality of kinds of feature values, and generates a second piece of distribution information on the basis of a kind of a second combination of feature values. Thereby, it becomes possible to display distribution information corresponding to a feature value aimed at by the user among the plurality of kinds of feature values.

In the case where the display control section has accepted a registration request for a partial region, the display control section saves information of a registered partial region indicating the partial region for which a registration request has been accepted, and further displays a distribution of the distribution information corresponding to the registered partial region. Thereby, a distribution of a partial region (ROI) already selected by the user can be announced.

The display control section displays each distribution of the distribution information by means of a representative value of a feature value. When the partial region is selected, the display control section displays a distribution of the selected partial region by means of a distribution having a spread specified by a feature value of the partial region. Thereby, the range of the distribution of the partial region can be easily viewed.

When a selection range is specified in the distribution information, the display control section displays a partial region that corresponds to a distribution of a partial region included in the selection range and is set in the image regarding pathology. Thereby, a partial region corresponding to a distribution of a partial region included in the selection range can be efficiently displayed.

A diagnosis support system including: a microscope; and software used for processing of a medical image corresponding to an object imaged by the microscope, in which the software (a generation program) causes a generation device to execute processing of, on the basis of feature values of partial regions set in an image regarding pathology, generating distribution information in which distributions of the partial regions on a feature space specified by feature values included in the partial regions are visibly arranged in the feature space while being associated with the partial regions. Thereby, the selection of a partial region having variations in characteristics can be supported.

The effects described in the present specification are merely examples and are not limitative ones, and there may be other effects.

The present technology can also have configurations like below.

(1)

A generation device including:

a generation section that, on the basis of feature values of partial regions set in an image regarding pathology, generates distribution information in which distributions of the partial regions on a feature space specified by feature values included in the partial regions are visibly arranged in the feature space while being associated with the partial regions.

(2)

The generation device according to (1), wherein the generation section generates the distribution information by, on the basis of feature values of a plurality of partial regions set in the image regarding pathology, arranging distributions of the plurality of partial regions respectively in the feature space.

(3)

The generation device according to (2), further including: a display control section that displays an image regarding pathology in which the plurality of partial regions are set and the distribution information.

(4)

The generation device according to (3), wherein when the partial region is selected, the display control section displays a distribution of the distribution information corresponding to the selected partial region.

(5)

The generation device according to any one of (1) to (4), further including: an acquisition section that acquires a feature value for the image regarding pathology; and a calculation section that calculates a feature value of the partial region on the basis of a feature value acquired by the acquisition section.

(6)

The generation device according to any one of (1) to (5), wherein the partial region has a plurality of kinds of feature values, and the generation section generates the distribution information on the basis of some kinds of feature values among the plurality of kinds of feature values.

(7)

The generation device according to (6), wherein the generation section generates a first piece of distribution information on the basis of a kind of a first combination of feature values among the plurality of kinds of feature values, and generates a second piece of distribution information on the basis of a kind of a second combination of feature values.

(8)

The generation device according to (3), wherein in a case where the display control section has accepted a registration request for a partial region, the display control section saves information of a registered partial region indicating the partial region for which a registration request has been accepted, and further displays a distribution of the distribution information corresponding to the registered partial region.

(9)

The generation device according to (3), wherein the display control section displays each distribution of the distribution information by means of a representative value of a feature values.

(10)

The generation device according to (9), wherein when the partial region is selected, the display control section displays a distribution of the selected partial region by means of a distribution having a spread specified by a feature value of the partial region.

(11)

The generation device according to (3), wherein when a selection range is specified in the distribution information, the display control section displays a partial region that corresponds to a distribution of a partial region included in the selection range and is set in the image regarding pathology.

(12)

A generation method including:

by a computer,

on the basis of feature values of partial regions set in an image regarding pathology, generating distribution information in which distributions of the partial regions on a feature space specified by feature values included in the partial regions are visibly arranged in the feature space while being associated with the partial regions.

(13)

A generation program for causing a computer to function as

a generation section that, on the basis of feature values of partial regions set in an image regarding pathology, generates distribution information in which distributions of the partial regions on a feature space specified by feature values included in the partial regions are visibly arranged in the feature space while being associated with the partial regions.

(14)

A diagnosis support system including: a microscope; and software used for processing of a medical image corresponding to an object imaged by the microscope,

wherein the software

causes a generation device to execute processing of, on the basis of feature values of partial regions set in an image regarding pathology, generating distribution information in which distribution of the partial regions on a feature space specified by feature values included in the partial regions are visibly arranged in the feature space while being associated with the partial regions.

### REFERENCE SIGNS LIST

100 GENERATION DEVICE
110 COMMUNICATION SECTION
120 INPUT SECTION
130 DISPLAY SECTION
140 STORAGE SECTION
141 PATHOLOGICAL IMAGE DB
142 PARTIAL REGION TABLE
143 CHARACTERISTIC DISTRIBUTION TABLE
150 CONTROL SECTION
151 ACQUISITION SECTION
152 CALCULATION SECTION
153 GENERATION SECTION
154 DISPLAY CONTROL SECTION
155 ANALYSIS SECTION

1. A generation device including:
a generation section that, on the basis of feature values of partial regions set in an image regarding pathology, generates distribution information in which distributions of the partial regions on a feature space specified by feature values included in the partial regions are visibly arranged in the feature space while being associated with the partial regions.

2. The generation device according to claim 1, wherein the generation section generates the distribution information by, on the basis of feature values of a plurality of partial regions set in the image regarding pathology, arranging distributions of the plurality of partial regions respectively in the feature space.

3. The generation device according to claim 2, further including: a display control section that displays an image regarding pathology in which the plurality of partial regions are set and the distribution information.

4. The generation device according to claim 3, wherein when the partial region is selected, the display control section displays a distribution of the distribution information corresponding to the selected partial region.

5. The generation device according to claim 1, further including: an acquisition section that acquires a feature value for the image regarding pathology; and a calculation section that calculates a feature value of the partial region on the basis of a feature value acquired by the acquisition section.

6. The generation device according to claim 1, wherein the partial region has a plurality of kinds of feature values, and the generation section generates the distribution information on the basis of some kinds of feature values among the plurality of kinds of feature values.

7. The generation device according to claim 6, wherein the generation section generates a first piece of distribution information on the basis of a kind of a first combination of feature values among the plurality of kinds of feature values, and generates a second piece of distribution information on the basis of a kind of a second combination of feature values.

8. The generation device according to claim 3, wherein in a case where the display control section has accepted a registration request for a partial region, the display control section saves information of a registered partial region indicating the partial region for which a registration request has been accepted, and further displays a distribution of the distribution information corresponding to the registered partial region.

9. The generation device according to claim 3, wherein the display control section displays each distribution of the distribution information by means of a representative value of a feature values.

10. The generation device according to claim 9, wherein when the partial region is selected, the display control section displays a distribution of the selected partial region by means of a distribution having a spread specified by a feature value of the partial region.

11. The generation device according to claim 3, wherein when a selection range is specified in the distribution information, the display control section displays a partial region that corresponds to a distribution of a partial region included in the selection range and is set in the image regarding pathology.

12. A generation method including:
by a computer,
on the basis of feature values of partial regions set in an image regarding pathology, generating distribution information in which distributions of the partial regions on a feature space specified by feature values included in the partial regions are visibly arranged in the feature space while being associated with the partial regions.

13. A generation program for causing a computer to function as
a generation section that, on the basis of feature values of partial regions set in an image regarding pathology, generates distribution information in which distributions of the partial regions on a feature space specified by feature values included in the partial regions are visibly arranged in the feature space while being associated with the partial regions.

14. A diagnosis support system including: a microscope; and software used for processing of a medical image corresponding to an object imaged by the microscope,
wherein the software
causes a generation device to execute processing of, on the basis of feature values of partial regions set in an image regarding pathology, generating distribution information in which distribution of the partial regions on a feature space specified by feature values included in the partial regions are visibly arranged in the feature space while being associated with the partial regions.
